(19) United States
(12) Patent Application Publication
(10) Pub. No.: US 2024/0161275 A1
(43) Pub. Date: May 16, 2024

(54) Indicating Baby torticollis using child growth monitoring system

(71) Applicant: UDISENSE INC., New York, NY (US)

(72) Inventors: Omri Levi, Givatayim (IL); Tomer Shmul, Tel-Aviv (IL); Felix Vilensky, Beer Ya'akov (IL); Samuel Hazak, Rehovot (IL); Amir Eitan, New York, NY (US); Roy Peleg, Tel-Aviv (IL); Shalom Elkayam, Rehovot (IL); Amit Ziv-Kenet, Haifa (IL); Tor Ivry, Rishon Lezion (IL)

(21) Appl. No.: 17/987,892

(22) Filed: Nov. 16, 2022

(51) Int. Cl. G06T 7/00; G06T 7/73; G06V 10/764; G06V 10/82; G06V 40/10; G06V 40/16; G06V 40/20; G16H 30/40

(52) U.S. Cl. CPC G06T 7/0012; G06T 7/73; G06V 10/764; G06V 10/82; G06V 40/103; G06V 40/168; G06V 40/23; G16H 30/40; G06T 2207/20081; G06T 2207/20084; G06T 2207/30201; G06T 2207/30232

(57) ABSTRACT

A method includes receiving a set of images of a child in a bed, the images acquired during a given period of time. A respective set of head postures of the child is classified from the set of images. Using the classified set of head postures, a head posture score of the baby is estimated. In response to the head posture score exceeding a predetermined threshold, a potentially abnormal child development issue is indicated and an action is taken upon the indication.

### FIELD OF THE INVENTION

The present invention relates generally to image processing, and particularly to apparatus, systems and methods for monitoring child growth.

### BACKGROUND

Systems and methods for monitoring child growth were previously proposed in the patent literature. International Patent Application WO 2021/146031, filed Dec. 21, 2020, whose disclosure is incorporated herein by reference, describes a method for photogrammetric measurement of child growth. An image is received, in an image coordinate frame, of a child lying on a bed, the bed covered by a patterned sheet bedsheet. The image is processed in order to identify the one or more patterns in the image and to match the one or more patterns identified in the image to one or more patterns in a pattern template corresponding to the patterned sheet bedsheet. A transformation is computed, based on the matched patterns, between the image coordinate frame and the template coordinate frame. A dimension of the child is measured by applying the computed transformation to the image of the child.

### SUMMARY

An embodiment of the present invention that is described hereinafter provides a method including receiving a set of images of a child in a bed, the images acquired during a given period of time. A respective set of head postures of the child is classified from the set of images. Using the classified set of head postures, a head posture score of the baby is estimated. In response to the head posture score exceeding a predetermined threshold, a potentially abnormal child development issue is indicated and an action is taken upon the indication.

In some embodiments, classifying a head posture of the child includes the steps of (i) processing one or more images in order to identify child body and head parts in the images, (ii) extracting body features from the one or more images, (iii) using the extracted body features, classifying a body posture, (iv) extracting head features from the one or more images, and (v) using the classified body posture and the extracted head features, classifying a head posture.

In some embodiments, using the classified body posture includes classifying body postures into one of six labeled classes of “back,” “belly,” “crawling,” “side,” “standing,” and “sitting,” and omitting from head posture classification head postures related to body postures of “side,” and “standing,” and “sitting.” In an embodiment, classifying the head posture includes classifying head postures into one of three labeled classes of “left,” “straight,” “and “right.”

In another embodiment, classifying body posture and head posture includes using a machine learning (ML) model that was trained using images of children in beds.

In yet another embodiment, classifying body posture and head posture includes using a machine learning (ML) model that was trained using images of children in beds.

In some embodiments, using a ML model to classify body posture includes using one of action recognition network (ARN) class and a classification network type of artificial neural networks (ANN).

In some embodiments, using a ML model to classify head posture includes using one of a multilayer perceptron (MLP) class and a convolutional neural network (CNN) class of artificial neural networks (ANN).

In an embodiment, extracting body features includes providing heatmaps of body joints.

In another embodiment, extracting head features include providing heatmaps including at least the nose, eyes and ears.

In yet another embodiment, extracting body features includes extracting skeletal features.

In an additional embodiment, extracting head features includes extracting at least one of facial features and features located at head circumference.

In some embodiments, the child is one of an infant and a toddler, and the bed is a crib.

In an embodiment, indicating of potentially abnormal child development issue includes indicating potential Torticollis.

In some embodiments, taking an action upon the indicated potentially abnormal child development issue includes sending an alert to a physician.

In some embodiments, the method further includes classifying from images a pattern of movement of the baby, generating a movement score, comparing the movement score to a threshold, and indicating a potentially abnormal child development issue based on the comparison.

In another embodiment, indicating a potentially abnormal child development issue using a movement score includes indicating potential Torticollis.

In some embodiments, the method further includes, in response to the head posture score, changing the period of time into a new period, classifying head posture based on images acquired only during the new period of time, and re-estimating accordingly the head posture score of the baby.

There is additionally provided, in accordance with another embodiment of the preset invention, a system including a camera and a processor. The camera is configured to acquire images of a child in a bed. The processor is configured to (i) receive a set of images of the child in the bed, the images acquired during a given period of time, (ii) classify from the set of images a respective set of head postures of the child, (iii) using the classified set of head postures, estimate a head posture score of the baby, and (iv) in response to the head posture score exceeding a predetermined threshold, indicate a potentially abnormal child development issue and taking an action upon the indication.

The present invention will be more fully understood from the following detailed description of the embodiments thereof, taken together with the drawings in which:

### BRIEF DESCRIPTION OF THE DRAWINGS

FIG. 1 is a block diagram that schematically illustrates a system for infant head posture estimation (HPE), in accordance with an embodiment of the invention;

FIG. 2 is a schematic top view of an infant image acquisition using a camera head mounted over an infant's crib, and preliminary analysis of the image, in accordance with an embodiment of the invention;

FIG. 3 is a flow chart that schematically illustrates a method for classifying body and head poses, in accordance with an embodiment of the invention;

FIGS. 4A and 4B are flow charts that schematically illustrate respective steps of classifying body and head poses using a trained machine learning (ML) model, in accordance with an embodiment of the invention; and

FIG. 5 is a flow chart that schematically illustrates a method for indicating a potentially abnormal child development issue and taking an action upon the indication, in accordance with an embodiment of the invention.

### DETAILED DESCRIPTION OF EMBODIMENTS

Torticollis is the Latin word for “twisted neck.” A stiff neck that is hard to turn and sometimes painful is referred to as torticollis, head tilt, or wryneck. The muscle that is affected by torticollis is the sternocleidomastoid muscle, which connects the head and neck to the breastbone. When this muscle is contracted, it causes a head tilt.

When torticollis is present in newborn babies, it's called infant torticollis or congenital muscular torticollis. Signs and Symptoms of Torticollis include:
The baby tilts his head to one side
The baby chin points the opposite way of the head tilt
The baby neck muscles seem tight
The baby favors looking over one shoulder instead of turning to look the other way
A breastfeeding baby may seem to favor one side or one breast
The baby shows frustration when he's unable to turn his head
The baby may develop a flat spot on his head from keeping his head in the same position when lying down
The baby may have a small lump, like a knot, on his neck on the side of the contracted muscle.

Currently assessments of Torticollis are done by a certified expert following a pediatrician recommendation, using the Muscle Function Scale and using an arthrodial goniometer as an instrument.

The present disclosure relates to using a combination of machine learning (ML) and computer vision methods with a baby monitor for assessing a baby's sleep posture patterns and utilizing this assessment for early detection and screening of motor development issues specifically torticollis and plagiocephaly.

Some embodiments of the present invention that are described hereinafter provide child growth monitoring systems and methods using trained machine learning models (ML), such as trained artificial neural networks (ANN) to classify body and head poses from photogrammetric measurements of a child.

In some embodiments, a processor of a system receives an input video and extracts still images from it. Each image is inputted to an ML based body and head pose detection algorithm. The processor running the algorithm outputs its estimations to body and head pose statistics block which summarizes the estimations and creates full body and head pose information on a daily, weekly and monthly basis.

For example, the disclosed technique may generate statistics of head pose over time, and compare the statistics to historical values or to a database of head pose statistics. Based on the comparison, a processor running a disclosed algorithm may output an HPE score. The HPE score can be divided into at least two regimes: one indicative head posture is within a normal range, and another indicative of a head posture of a potentially abnormal tendency (e.g., to the right or to the left). A user receiving the indication, such as a medical doctor in the community receiving an alert of an abnormal score electronically filed into a medical file of the baby, may ask in response to the alert (that the system provided), to examine the baby.

To acquire video and/or images, a camera, such as a dedicated monitoring camera or a handheld device (for example, a smartphone camera) is included, that, for example, captures images of a child (e.g., an infant or a toddler) lying in a bed (e.g., a crib).

The disclosed solutions give parents statistics of the body and head poses, and parents can use this information to further check if there is a growth issue (such as Torticollis). In some embodiments, the processor may automatically identify the locations of joints and other key landmarks in the child's body. The processor then estimates and classifies body and head postures of the child. Such classified body and head poses can alert a parent of a child development problem (e.g., of Torticollis).

The processor extracts body joints and head key-points from the input image using a skeleton neural network, and the user does not need to mark the joints and key-points manually.

In one embodiment, to identify Torticollis, the disclosed technique estimates head pose by detecting if the baby's head is at the center or turning to the left or to the right with respect to the body (e.g., providing coarse yaw classification). After gathering sufficient statistics, the processor can indicate if there is a head pose anomaly.

Typically for head pose classification, the technique described in the embodiment includes the steps of training an ANN using frames from RGB and IR sensors with subjects that are babies or infants in various complex body poses, with their faces capture only a fraction of the frame size.

In some embodiments, classification of the head pose comprises building a classifier with at least three head pose labels: left, center and right. This solution also incorporates learning-based methods which detects the baby's head and body bounding boxes, extracts face key-points and body pose. A processor combines this information all together, analyzes it in either a learning-free (i.e., without training) and learning manner (i.e., relaying on training) and classifies the baby's head pose.

In an exemplary embodiment of the disclosed technique, a baby monitor is incorporated with a computer-implemented method of determining a state of a baby, the method comprising: receiving a video of the baby captured by a camera capable of imaging body features; receiving information from different sensors; analyzing the image to receive an indicator to a clinical parameter of the baby; and fusing the indicator with data sources and prior information of the baby (for example—age, gender, general location, culture, height weight head circumference, etc.) to obtain a pose score assessment of the baby. The method can further comprise determining and taking an action in response to the score exceeding a predetermined threshold. Within the method, the action optionally affects the behavior of the system. Within the method, the behavior optionally relates to changing parameters in accordance with room conditions and baby behavior. Within the method, the action is optionally collecting information to be provided to a medical provider or insurer and giving back suggestions to the parent. The method can further comprise collecting more information related to the case and issuing a warning to the parent if the score levels exceed a threshold. The method can further comprise: receiving data from an additional source; and analyzing the data to obtain an additional indicator; and fusing the additional indicator with the telemetry information and the indicator to obtain the assessment. Within the method, the additional source is optionally an image capture device or a voice capture device and further comprises analyzing an image captured by the image capture device or voice captured by the voice capture device. Within the method, the additional source is at least one item selected from the group consisting of sleep analytics data of the baby, vitals data (respiratory, heart rate, SpO2), and a wearable device. The method can further comprise adapting parameters or thresholds from a behavior of the baby over time and using the parameters or thresholds as learned from multiple babies over big data in obtaining a new head posture score assessment.

In another example, in response to the head posture score, the processor can change the period of time into a new period, classify head posture based on images only during the new period of time (or use already classified postures if new period is included in the given period of time), and re-estimate the head posture score of the baby. Using this approach, an algorithm that the processor runs optimizes the period of time into one deemed most relevant one and provides the indication based on analysis of the data from the deemed most relevant period of time. Other events may trigger such re-estimation, such as periodic re-estimation using a new period of time (e.g., latest three months, latest half year, etc.).

In other embodiments, a processor can use the disclosed baby monitoring system and software to analyze additional information apart of visual one (e.g., video of the baby's body, head posture and baby movement). The system may include sensors—microphones, radars patterned light sensors, IMU, etc. The software may include algorithms for sleep analysis, vitals detection, cry detection, pose detection, etc. Form data can be utilized, as field by users via an application provided with the system. Big data—metadata collected from other baby monitors can be utilized. Other bedside devices—movement detection pad, light and sound devices, etc. Data is stored over time and can also be used in fusion with other information. This data and system can be fused and used to take action, such as:
Analyzing the data and showing it in an application dashboard.
Giving tips and suggestions to the parent automatically.
Initiating a more complex test using light and sound stimulation to the baby.
Changing monitoring levels, and thresholds.
Sending/saving video to pediatrician or parent.

Finally, in yet another embodiment, the disclosed technique provides baby movement scoring that can also be used as indicator of potentially abnormal child development issue. For example, the disclosed technique uses an observation that difficulties in baby movement in crib may indicate that the aforementioned sternocleidomastoid muscle, which connects the head and neck to the breastbone, is not properly functioning, thereby causing Torticollis. To this end, the disclosed technique generates from the baby images a movement score, e.g., head movement score or whole-body movement score. A processor compares such movement with a database of scored movements, to indicate difficulty of a baby to change posture in its crib. For example, by comparing the movement score to a threshold, the processor can indicate of potentially abnormal child development. Specifically, based on the comparison difficulty in changing a head posture by moving the head in crib, the technique can indicate of a developing issue of Torticollis.

### System Description

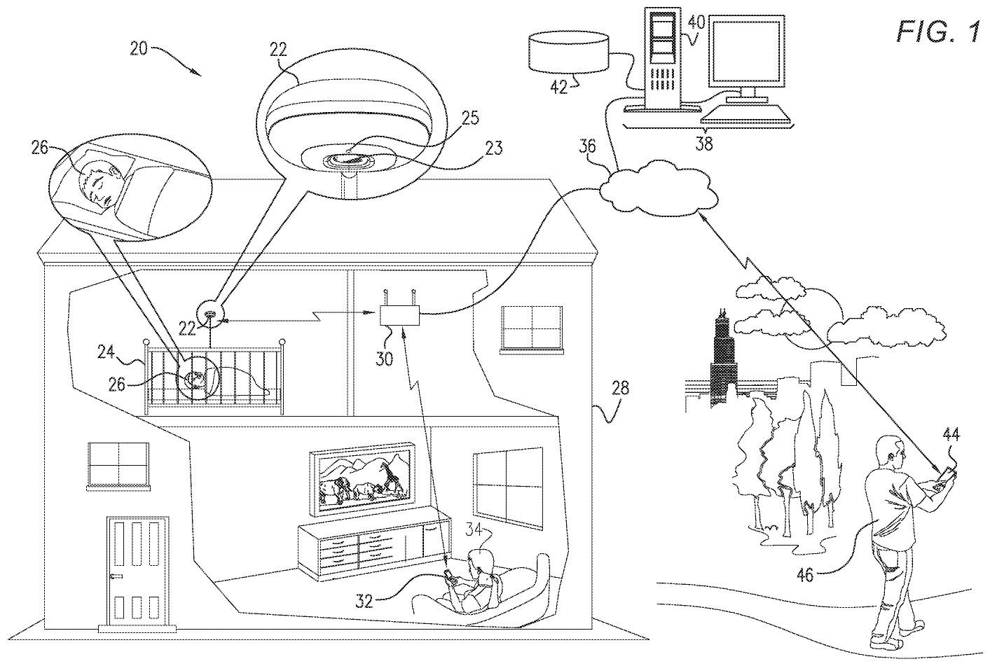

FIG. 1 is a block diagram that schematically illustrates a system 20 for infant head pose estimation (HPE), in accordance with an embodiment of the invention. System 20 comprises a monitoring camera head 22, which is mounted above a crib 24, in which an infant 26 is sleeping in a residence 28. Alternatively, camera head 22 may be mounted in another suitable location in proximity to the crib, mounted on a wall or tabletop, for example. Further alternatively, as noted earlier, some of the functionalities of camera head 22 may be carried out using a handheld camera (not shown) with suitable communication capabilities, such as the camera in a smartphone.

For purposes of image capture in the pictured system, an infrared (IR) light-emitting diode (LED) 25 on the lower side of camera head 22 illuminates the sleeping infant 26. A diffuser can be used to spread the infrared light uniformly across the crib. Camera head 22 also comprises an infrared-sensitive image sensor 23. The resolution and sensitivity of image sensor 23 can be optimized for night conditions, and specifically for the wavelength range of LED 25. Further details of camera head 22, including its internal components and modes of operation, are described in PCT International Publication WO 2017/196695, whose disclosure is incorporated herein by reference. This PCT publication also describes different ways of mounting the camera head above or alongside the crib.

Camera head 22 transmits digitized streaming video, and possibly other signals, as well, over a local network to a router 30, typically via a wireless local area network (LAN) link, such as a Wi-Fi connection, or a wired link, such as an Ethernet connection. Camera head 22 transmits the digitized video data in packets that are addressed so that router 30 forwards the video packets to either or both of a local client device 32 on the local network and a remote server 38 (e.g., cloud server) via a public network 36, such as the Internet. Client device 32 typically comprises a smartphone, tablet or personal computer, which enables a caregiver 34 in another room of residence 28 to monitor infant 26, even when there is no Internet connection available. Server 38 makes video images and other data available to authorized remote client devices 44, thus enabling a caregiver 46 to monitor infant 26 at any location where there is access to public network 36. The Wi-Fi or other local network connection provides reliable video streaming from camera head 22 to client device 32 with high bandwidth and low latency, even if the external Internet connection is not working. As long as the Internet is connected, however, the video stream is also transmitted to server 38 for purposes of analysis and retransmission.

Server 38 typically comprises a general-purpose computer, comprising a processor 40 and a memory 42, which receives, stores and analyzes images from camera head 22 in residence 28 and similarly from other cameras in other residences (not shown). In the present embodiment, processor 40 analyzes the images in order to make photogrammetric measurements of infant 26, as described further hereinbelow.

Processor 40 typically performs these functions under the control of software, which may be downloaded to server 38 in electronic form, over a network, for example, as well as stored on tangible, non-transitory computer-readable media, such as magnetic, optical or electronic memory media. Alternatively or additionally, some or all of these processing, measurement and monitoring functions may be performed locally, for example by a microprocessor in camera head 22 and/or by suitable application software running on processors in client devices 32 and/or 44.

### Image Acquisition and Analysis

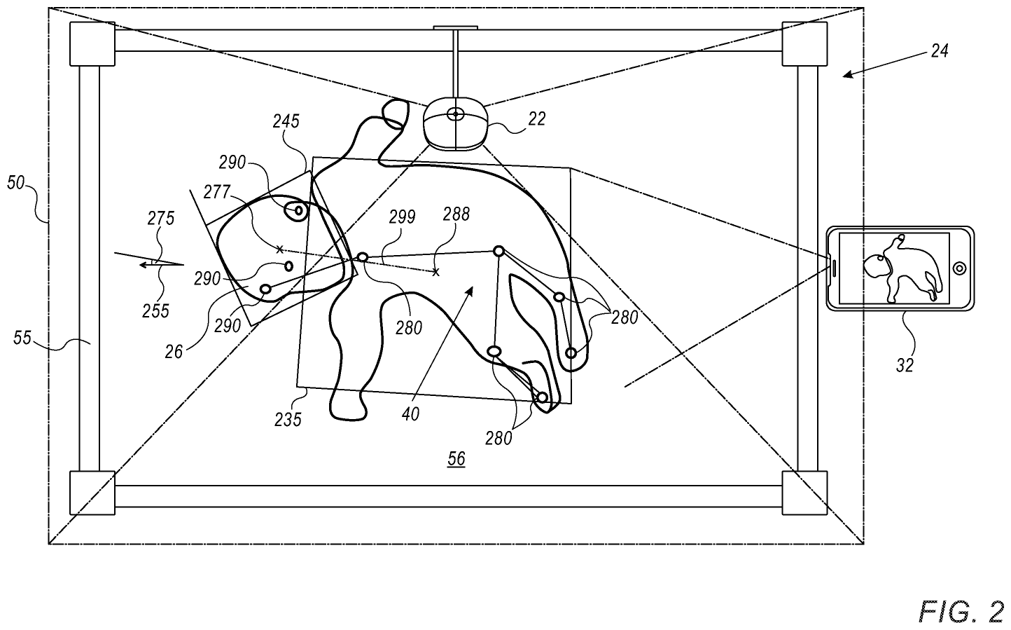

FIG. 2 is a schematic top view of an infant 26 image acquisition using a camera head 22 mounted over an infant's crib 24, and preliminary analysis of the image, in accordance with an embodiment of the invention.

In the pictured embodiment, monitoring camera head 22 stands against a wall over crib 24. Camera head 22 is held, for example, at the end of an arm at the upper end of a tripod mount behind crib 24, at the midpoint of the long side of the crib. Camera head 22 in this embodiment is positioned and adjusted so that the camera head has a field of view (FOV) 50 from a perspective that encompasses all or most of the area of crib 24, including the border crib frame 55 of a mattress 56. This perspective provides server 38 with image information that can be analyzed conveniently and reliably.

Processor 40 of server 38 runs an algorithm that receives an image with field of view 50 and an image axis 255 of infant 26, processor 40 then runs an algorithm that detects head and body and outputs body and head bounding boxes 235 and 245, respectively, and outputs bounding boxes with image axis 255. In an optional preliminary analysis step, the processor aligns the center 277 of the head bounding box 245 and the center 288 of the body bounding box 235 and rotates the image by an angle 275 such that a line 299 between the centers is vertical and the head is at the top (“top” assumed herein as left side of FIG. 2). Such alignment step saves a need to use an augmented set of training images during classification.

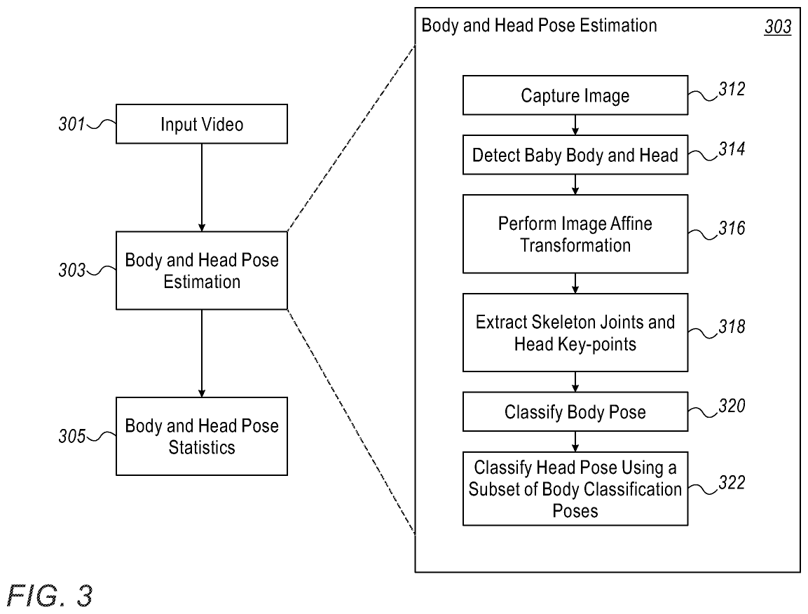

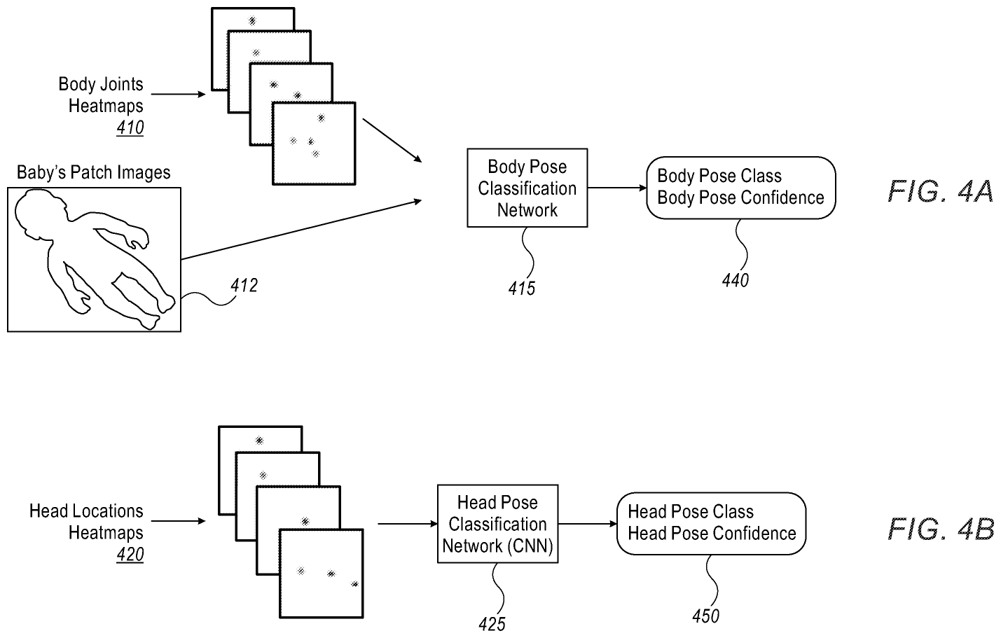

The processor then extracts body joints 280 coordinates and head key-points coordinates 290. (Head key-points may include eyes, ears, and nose.) To do this, the disclosed technique uses a skeleton network model 285 to generate coordinates of body and head joints (280, 290). A method of extraction of joints using a skeleton model (method called therein, “key-point detection”) is provided by Wang et al., in a paper, “Deep High-Resolution Representation Learning for Visual Recognition,” published in IEEE TRANSACTIONS ON PATTERN ANALYSIS AND MACHINE INTELLIGENCE, MARCH 2020, (arXiv:1908.07919). This information is subsequently processed and used with an ML model for classification of body and head postures as described in FIGS. 3 and 4.

For the purpose of extracting body joints 280 coordinates and head key-points coordinates 290 a smaller field of view than FOV 50 may be used.

In another an optional embodiment, the camera head may be mounted in any other suitable location in proximity to crib 24; or a handheld camera may be used, as noted above. For example, as shown in FIG. 2, a smartphone, such as client device 32, may be used to capture images of crib 24 and infant 26. In the pictured example, client device 32 captures an image from a location on the short side of crib 24, from a high angle. The methods of transformation and measurement that are described herein may be applied to such images, as well.

### Method for Baby Posture Detection Using Machine Learning

FIG. 3 is a flow chart that schematically illustrates a method for classifying body and head poses, in accordance with an embodiment of the invention. The process is divided into three blocks 301, 303 and 305. Block 301 handles the step of receiving an input video. Block 303 is concerned with estimation of body and head postures from a particular set of images, the images derived from the captured video. The particular estimation includes substantial uncertainty, or given level of confidence that is too low to indicate by itself an anomaly. In a block 305, the processor running the algorithm outputs its estimations to body and head pose statistics block which summarizes the estimations and creates full body and head pose information on a daily, weekly and monthly basis. The accumulated estimation, such as on a monthly basis, is expected to have far higher accuracy.

As seen in FIG. 3, block 303 includes steps 312-322, that in one embodiment, processor 40 of server 38 (FIG. 1)

TABLE 1

| Step | | Step | | |
|---|---|---|---|---|
| # | Step name | description | Inputs | Outputs |
| 312 | Image | Extract of an | 1. Input image | — |
| | capture | input image | with baby in | |
| | | from video | crib | |
| 314 | Body and | Locate baby's | 1. Full input | 1. Baby's body |
| | head | body and head | image | and head |
| | detection | on the input | | bounding |
| | | image. | | boxes with |
| | | | | prediction |
| | | | | confidence |
| | | | | values. |
| 316 | Image | Calculate the | 1. Full input | 1. Affine |
| | affine | affine | image | transformation |
| | trans- | transformation | 2. Baby's body | matrix. |
| | formation | such that the | and head | 2. Warped |
| | | baby's head is | bounding boxes | image with |
| | | aligned to the | | baby's head |
| | | image top. | | aligned to |
| | | Apply | | image top |
| | | transformation | | 3. Warped |
| | | on input image | | baby's body |
| | | and baby's | | and head |
| | | body and head | | bounding |
| | | bounding | | boxes |
| | | boxes. | | |
| 318 | Skeleton | Extract body | 1. Warped image | 1. Baby's body |
| | joints | joints and | 2. Warped baby's | joints |
| | extraction | head key- | body bounding | coordinates |
| | | points. Roll | box. | and head |
| | | back the | 3. Inverse affine | key-points |
| | | joints and | matrix. | coordinates |
| | | key-points | | with |
| | | coordinates to | | prediction |
| | | the full image | | confidence |
| | | coordinate | | values (for |
| | | system using | | each joint/ |
| | | the inverse | | key-point). |
| | | affine matrix. | | Coordinates |
| | | | | in the full |
| | | | | image |
| | | | | coordinates |
| | | | | system. |
| 320 | Body pose | Classify | 1. Body joints | 1. Baby's body |
| | classi- | baby's body | and head key- | pose class |
| | fication | pose according | points | prediction |
| | | to body joints | coordinates | with |
| | | and head key- | and | confidence |
| | | points | confidences. | value. |
| | | coordinates. | | |
| | | Create joints | | |
| | | and key-points | | |
| | | heatmaps and | | |
| | | classify body | | |
| | | pose. | | |
| 322 | Head pose | Classify | 1. Head key- | 1. Baby's head |
| | classi- | baby's head | points | pose class |
| | fication | pose according | coordinates | prediction |
| | | to head key- | and | with |
| | | points | confidences. | confidence |
| | | coordinates | 2. Head bounding | value. |
| | | and baby's | box. | |
| | | body pose | 3. Body pose | |
| | | prediction. | prediction. | |

In above step 314 of body and head detection, the processor receives a frame and sends it to an object detection network, which outputs the baby's body location (bounding box) and baby's head location (bounding box) including confidence values for both predictions. One useful object detection network, brought only by way of example since other object detection networks that can be used, is called YOLOX and it was trained using a custom object detection dataset by the inventors. YOLOX is described by Ge et al. in a paper titled, “YOLOX: Exceeding YOLO Series in 2021,” published as arXiv:2107.08430, and is incorporated herein by reference.

In above optional step 316, the affine transformation (that may be viewed as an inhomogeneous linear transformation defined by y=Ax+b), the processor calculates the affine transformation such that the baby's head is aligned to the image top, as described in FIG. 2.

In above step 318, of skeleton joints extraction, the processor extracts body joints 280 and head key-points 290. In a further optional portion of step 318 (i.e., if optional step 316 is used), the processor rolls back the joints and key-points coordinates to the full image coordinate system using the inverse affine matrix.

As a preprocessing step of the above step 320, processor 40 generates from locations 280 and head key-points 290 2D heatmaps, that are subsequently inputted to an ML model, as described in FIGS. 4A and 4B. For example, for each joint, this pre-processing stage provides heatmap from the joint's location and confidence value.

Optionally, processor 40 may use locations 280 of joints and other key landmarks in the image of the body of infant 26, in constructing a geometrical skeleton 295. (The term “skeleton,” as used in the context of the present description and in the claims, refers to a geometrical construct connecting joints and/or other landmarks identified in an image and does not necessarily to correspond to the infant's physiological skeleton.) In some examples, locations 280 the bottom of the infant's neck, the center of the hip, the knees, and the bottoms of the feet. Alternatively or additionally, other points may be identified. The processor can extract the skeleton autonomously, using methods of human pose estimation that are known in the art. Deep learning approaches may be effectively applied for this purpose, for example as described by Sun et al., in a paper titled, “Deep High-Resolution Representation Learning for Human Pose Estimation,” that appeared in Proceedings of the IEEE/CVF Conference on Computer Vision and Pattern Recognition (CVPR), 2019, pages 5693-5703, and which is incorporated herein by reference.

In step 320, the processor runs a disclosed algorithm that classifies the body pose (also called “postures”) into one of six labeled classes of “back,” “belly,” “crawling,” “side,” “standing,” and “sitting,” and omitting from head posture classification head postures related to body postures of “side,” and “standing,” and “sitting.”

In step 322, the disclosed algorithm that classifies the head pose only for the following body poses: back, belly and crawling. Sitting and Standing poses are not considered for head pose classification. “Side” body pose may be not considered as well, or when body side pose is inputted the head pose is classified as “center” (e.g., neutral classification).

FIGS. 4A and 4B are flow charts that schematically illustrate respective steps of classifying body and head poses using a trained machine learning (ML) model, in accordance with an embodiment of the invention.

ML model 415 may be used to classify body posture (also called “pose”). The ML model receives as one input heatmaps 410 for a pose classification network. As another input, ML model 415 receives baby patch images 412.

In the example of FIG. 4A, ML model 415 is pose classification network based on a combination of an action recognition network (ARN) class and a classification network type (e.g., the “EfficientNet” solution referenced below) of artificial neural networks (ANN). ARN is described by Duan et al. in a paper titled, “Revisiting Skeleton-based Action Recognition,” published in Proceedings of the IEEE/CVF Conference on Computer Vision and Pattern Recognition (CVPR), 2022, pages 2969-2978. As opposed to the mentioned network from the literature, the disclosed technique does not use temporal information as the purpose herein is to classify body pose and not an action.

EN model is described by Tan and Le in a paper titled, “EfficientNet: Rethinking Model Scaling for Convolutional Neural Networks,” published in Proceedings of the 36th International Conference on Machine Learning, PMLR 97, pages 6105-6114, 2019. The ARN and EN papers are incorporated herein by reference.

In FIG. 4A, ML model 415 optionally uses both the ARN and EN solutions by concatenating hidden layers from each network which will pass through mutual classification module.

In the given example, ML model 415 classifies body postures into one of six labeled classes (440) of “back,” “belly,” “crawling,” “side,” “standing,” and “sitting,” and omitting from head posture classification head postures related to body postures of “side,” and “standing,” and “sitting.”

FIG. 4A is brought by way of example. Note, in other embodiments, ML model 415 may be standalone ARN or an EN, working respectively, with one type of input (heatmaps or images).

In FIG. 4B, an ML model 425 may be used to classify head posture (also called “pose”). The method according to claim 1, wherein ML models to classify head posture comprises using one of a multilayer perceptron (MLP) class and a convolutional neural network (CNN) class of artificial neural networks (ANN). A relevant CNN is described by Gupta et al. in a paper titled, “Nose, Eyes and Ears: Head Pose Estimation by Locating Facial Keypoints,” published in ICASSP 2019, submitted December 2018, arXiv:1812.00739, and incorporated herein by reference. MLP architectures are described in ML textbooks. One input for an MLP based head pose classification network is heatmaps of head landmarks (e.g., pictorial relative locations of nose, eyes and ears). Instead of MLP one can use neural network which gets list of joints/key-points (with locations (x, y) and confidence values). The network pre-processes the joints: one option for the pre-processing step is normalizing the locations of joints to have a common coordinate system, other option is to create the aforementioned heatmaps from it. The processor classifies the head posture into one of three labeled classes (450) of “left,” “straight,” “and “right.” The processor runs a disclosed algorithm, where model 425 classifies (450) the head pose only for the following body poses are classified (440): back, belly and crawling. Sitting and Standing poses are not relevant for our head pose prediction. and assuming “straight” (also called “center”) head pose for “side” body pose.

### Method of Indicating Baby Torticollis Using Child Growth Monitoring System

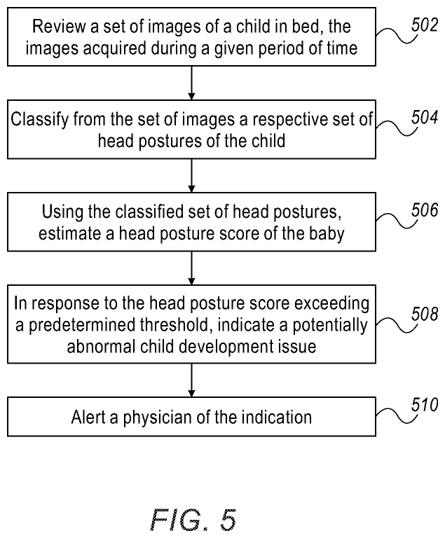

FIG. 5 is a flow chart that schematically illustrates a method for indicating a potentially abnormal child development issue and taking an action upon the indication, in accordance with an embodiment of the invention. The algorithm according to the presented embodiment carries out a process that begins with system 20 receiving a set of images of a child in a bed, the images acquired in batches (e.g., daily batches) during a given period of time (e.g., over a month), at images acquisition step 502.

A processor of system 20 classifies from the set of images a respective set of head postures of the child, e.g., as described above in FIG. 4, at head posture classification step 504.

At the end of the time period, at head posture scoring step 506, using the classified set of head postures, the processor (e.g., processor 40) of system 20 estimates a head posture score of the baby.

In response to the head posture score exceeding a predetermined threshold, the processor indicates a potentially abnormal child development issue, at indication step 508.

Finally, at an alerting step 510, the system takes an action upon the indication, such as processor 40 sending an alert to a physician of the child.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method, comprising:
receiving a set of images of a child in a bed, the images acquired during a given period of time;
classifying from the set of images a respective set of head postures of the child;
using the classified set of head postures, estimating a head posture score of the baby; and
in response to the head posture score exceeding a predetermined threshold, indicating a potentially abnormal child development issue and taking an action upon the indication.

2. The method according to claim 1, wherein classifying a head posture of the child comprises the steps of:
processing one or more images in order to identify child body and head parts in the images;
extracting body features from the one or more images;
using the extracted body features, classifying a body posture;
extracting head features from the one or more images; and
using the classified body posture and the extracted head features, classifying a head posture.

3. The method according to claim 2, wherein using the classified body posture comprises classifying body postures into one of six labeled classes of “back,” “belly,” “crawling,” “side,” “standing,” and “sitting,” and omitting from head posture classification head postures related to body postures of “side,” and “standing,” and “sitting.”

4. The method according to claim 2, wherein classifying the head posture comprises classifying head postures into one of three labeled classes of “left,” “straight,” “and “right.”

5. The method according to claim 2, wherein classifying body posture and head posture comprises using a machine learning (ML) model that was trained using images of children in beds.

6. The method according to claim 5, wherein using a ML model to classify body posture comprises using one of action recognition network (ARN) class and a classification network type of artificial neural networks (ANN).

7. The method according to claim 5, wherein using a ML model to classify head posture comprises using one of a multilayer perceptron (MLP) class and a convolutional neural network (CNN) class of artificial neural networks (ANN).

8. The method according to claim 2, wherein extracting body features comprises providing heatmaps of body joints.

9. The method according to claim 2, wherein extracting head features comprises providing heatmaps comprising at least the nose, eyes and ears.

10. The method according to claim 2, wherein extracting body features comprises extracting skeletal features.

11. The method according to claim 2, wherein extracting head features comprises extracting at least one of facial features and features located at head circumference.

12. The method according to claim 1, wherein the child is one of an infant and a toddler, and the bed is a crib.

13. The method according to claim 1, wherein indicating of potentially abnormal child development issue comprises indicating potential Torticollis.

14. The method according to claim 1, wherein taking an action upon the indicated potentially abnormal child development issue comprises sending an alert to a physician.

15. The method according to claim 1, and comprising classifying from images a pattern of movement of the baby, generating a movement score, comparing the movement score to a threshold, and indicating a potentially abnormal child development issue based on the comparison.

16. The method according to claim 15, wherein indicating a potentially abnormal child development issue using a movement score comprises indicating potential Torticollis.

17. The method according to claim 1, and comprising, in response to the head posture score, changing the period of time into a new period, classifying head posture based on images acquired only during the new period of time, and re-estimating accordingly the head posture score of the baby.

18. A system, comprising:
a camera configured to acquire images of a child in a bed; and
a processor, which is configured to:
receive a set of images of the child in the bed, the images acquired during a given period of time;
classify from the set of images a respective set of head postures of the child;
using the classified set of head postures, estimate a head posture score of the baby; and
in response to the head posture score exceeding a predetermined threshold, indicate a potentially abnormal child development issue and taking an action upon the indication.

19. The system according to claim 18, wherein the processor is configured to classify a head posture of the child by performing at least the steps of:
processing one or more images in order to identify child body and head parts in the images;
extracting body features from the one or more images;
using the extracted body features, classifying a body posture;
extracting head features from the one or more images; and
using the classified body posture and the extracted head features, classifying a head posture.

20. The system according to claim 19, wherein the processor is configured to use the classified body posture by classifying body postures into one of six labeled classes of “back,” “belly,” “crawling,” “side,” “standing,” and “sitting,” and omitting from head posture classification head postures related to body postures of “side,” and “standing,” and “sitting.”

21. The system according to claim 19, wherein the processor is configured to classify the head posture by classifying head postures into one of three labeled classes of “left,” “straight,” “and “right.”

22. The system according to claim 19, wherein the processor is configured to classify body posture and head posture by using a machine learning (ML) model that was trained using images of children in beds.

23. The system according to claim 22, wherein the processor is configured to use a ML model to classify body posture by using one of action recognition network (ARN) class and a classification network type of artificial neural networks (ANN).

24. The system according to claim 22, wherein the processor is configured to use a ML model to classify head posture by using one of a multilayer perceptron (MLP) class and a convolutional neural network (CNN) class of artificial neural networks (ANN).

25. The system according to claim 19, wherein the processor is configured to extract body features by providing heatmaps of body joints.

26. The system according to claim 19, wherein the processor is configured to extract head features by providing heatmaps comprising at least the nose, eyes and ears.

27. The system according to claim 19, wherein the processor is configured to extract body features by extracting skeletal features.

28. The system according to claim 19, wherein the processor is configured to extract head features by extracting at least one of facial features and features located at head circumference.

29. The system according to claim 18, wherein the child is one of an infant and a toddler, and the bed is a crib.

30. The system according to claim 18, wherein the processor is configured to indicate of potentially abnormal child development issue by indicating potential Torticollis.

31. The system according to claim 18, wherein the processor is configured to take an action upon the indicated potentially abnormal child development issue by sending an alert to a physician.

32. The system according to claim 18, wherein the processor is further configured to classify from images a pattern of movement of the baby, generate a movement score, compare the movement score to a threshold, and indicate a potentially abnormal child development issue based on the comparison.

33. The system according to claim 32, wherein the processor is configured to indicate a potentially abnormal child development issue using a movement score by indicating potential Torticollis.

34. The system according to claim 18, wherein the processor is further configured to, in response to the head posture score, change the period of time into a new period, classify head posture based on images acquired only during the new period of time, and re-estimate accordingly the head posture score of the baby.
